(19) United States
(12) Patent Application Publication
(10) Pub. No.: US 2025/0222237 A1
(43) Pub. Date: Jul. 10, 2025

(54) Fluid Path Optimization in Catheter Insertion Systems

(71) Applicant: Bard Access Systems, Inc., Salt Lake City, UT (US)

(72) Inventors: Joe Spataro, Cottonwood Heights, UT (US); Austin J. Mckinnon, Herriman, UT (US); Bryan Haymond, Emigration Canyon, UT (US)

(21) Appl. No.: 19/094,613

(22) Filed: Mar. 28, 2025

Related U.S. Application Data
(62) Division of application No. 17/557,924, filed on Dec. 21, 2021, now Pat. No. 12,263,316.
(60) Provisional application No. 63/128,677, filed on Dec. 21, 2020.

(51) Int. Cl. A61M 25/06; A61M 25/01

(52) U.S. Cl. CPC A61M 25/0693; A61M 2025/0183

(57) ABSTRACT

A method of inserting a catheter into a vasculature of a patient can include disposing the catheter into a housing. The housing can include a blood flash indicator, having a syringe plunger fixed to the housing and a syringe barrel slidable relative to the syringe plunger. The method can further include positioning the housing adjacent the vasculature, inserting a needle into the housing and through a lumen of the catheter until a distal tip of the needle extends from a distal end of the catheter, and accessing the vasculature via the distal tip of the needle. The method can further include sliding the syringe barrel in a proximal direction relative to the syringe plunger to create a vacuum in a lumen of the needle to thereby draw blood through the lumen of the needle and into the syringe barrel.

### PRIORITY

This application is a division of U.S. patent application Ser. No. 17/557,924, filed Dec. 21, 2021, now U.S. Pat. No. 12,263,316, which claims the benefit of priority to U.S. Provisional Application No. 63/128,677, filed Dec. 21, 2020, each of which is incorporated by reference in its entirety into this application.

### SUMMARY

Briefly summarized, embodiments disclosed herein are directed to fluid path optimization in catheter insertion systems, and associated methods thereof. When placing elongate catheters, such as central venous catheters (CVC), rapid insertion central catheters (RICC), or the like, advanced insertion systems are desirable. These insertion systems can include housings, needles, guidewires, dilators, or blood flash indicators configured to access the vasculature, confirm correct vascular access, dilate the insertion site and place the catheter. Advantageously, the insertion systems can contain the above structures within an enclosed environment to reduce the repeated insertion and removal of multiple devices, and mitigate the introduction of pathogens.

The sequential nature of the tools within these insertion systems can result in a long and convoluted pathways for the blood flow before reaching the blood flash indicator. For example, a needle disposed within a lumen of the catheter, requires blood to flow to at least a proximal end of the catheter before exiting and being observed. Further, blood flash indicators can be situated towards a distal end of the insertion device, proximate the insertion site for ease of observation. As such the blood flow would have to travel twice the length of the catheter before being observed.

Such elongate flow pathways can lead to various problems. For example, the longer flow path requires increased volumes of blood to flow through the system before reaching the blood flash indicator and being observed. Such elongate flow paths can obscure the nature of the pulsatile flow, obfuscating the difference between arterial or venous flow. The delay caused by the increase travel time can lead to a user continuing with insertion despite having accessed the vasculature, resulting in “backwalling” the vessel, i.e. inserting the needle through a far wall of the vessel. Where blood flash systems include a vacuum to draw the blood flow, the convoluted flow path can reduce or compromise the force of the vacuum. Lastly, elongate blood flow pathways can have an increased risk of the blood clotting prior to reaching the blood flash indicator. Embodiments disclosed herein are directed to resolving the aforementioned problems.

Disclosed herein is a catheter insertion system including, a catheter defining a catheter lumen and extending along a longitudinal axis, a needle defining a needle lumen and disposed within a portion of the catheter lumen, a housing supporting one of the catheter or the needle, and a blood flash indicator in fluid communication with the needle lumen, the blood flash indicator including a plunger fixedly attached to the housing and a syringe barrel slidably engaged with the plunger along the longitudinal axis.

In some embodiments, the syringe barrel is configured to slide proximally relative to one of the plunger or the housing to create a vacuum and draw a blood flow through the needle lumen and into the syringe barrel. The syringe barrel is formed of a transparent material to allow for observation of a blood color or pulsatile flow. The syringe barrel is supported by a cradle including a gripping feature and configured to facilitate sliding of the syringe barrel along the longitudinal axis. The housing includes a needle interface defining a needle channel and a blood flash channel, the needle channel configured to receive a portion of the needle extending therethrough and the blood flash channel providing fluid communication between the needle channel and the syringe barrel.

In some embodiments, the needle includes a notch extending through a wall of the needle to provide fluid communication between the needle lumen and the needle channel. The needle channel includes a first O-ring disposed annularly about the needle and disposed distally of the needle notch, and a second O-ring disposed annularly about the needle and disposed proximally of the needle notch, the first O-ring and the second O-ring each extending between an outer surface of the needle and an inner surface of the needle channel to provide a fluid-tight seal therebetween. The needle is slidably engaged with the needle channel. In some embodiments, a portion of the blood flash channel is formed of a flexible tube. The catheter is a central venous catheter or a rapid insertion central catheter.

Also disclosed is a method placing a catheter including, accessing a vasculature with a needle, defining a needle lumen and supported by a housing, sliding a syringe barrel proximally along a longitudinal axis relative to the housing, creating a vacuum within the syringe barrel, and drawing a blood flow through the needle lumen and into the syringe barrel.

In some embodiments, the method further includes a plunger slidably engaged with the syringe barrel, the plunger fixedly attached to the housing to prevent any relative longitudinal movement therebetween. In some embodiments, the method further includes a cradle coupled with the syringe barrel and including a gripping feature. In some embodiments, the method further includes observing a blood flow color or pulsatile flow within the syringe barrel, the syringe barrel formed of a transparent material. In some embodiments, the housing includes a needle interface defining a needle channel and a blood flash channel, the needle channel configured to receive a portion of the needle extending therethrough and the blood flash channel providing fluid communication between the needle channel and the syringe barrel. The needle includes a notch extending through a wall of the needle to provide fluid communication between the needle lumen and the needle channel.

In some embodiments, the needle channel includes a first O-ring disposed annularly about the needle and disposed distally of the needle notch, and a second O-ring disposed annularly about the needle and disposed proximally of the needle notch, the first O-ring and the second O-ring each extending between an outer surface of the needle and an inner surface of the needle channel to provide a fluid-tight seal therebetween. The needle is slidably engaged with the needle channel. In some embodiments, a portion of the blood flash channel is formed of a flexible tube. The catheter is a central venous catheter or a rapid insertion central catheter.

Also disclosed is a catheter placement system including, a catheter defining a catheter lumen and extending along a longitudinal axis, a needle defining a needle lumen and extending through a portion of the catheter lumen, a housing supporting one or both of the catheter and the needle, and including a needle interface piece to provide fluid communication between the needle lumen and a blood flash channel, and a blood flash indicator in fluid communication with the blood flash channel at a point distal of a proximal end of the needle, the blood flash indicator including a syringe barrel fixedly attached to the housing and a plunger slidably engaged with the syringe barrel between proximal position and a distal position.

In some embodiments, the plunger is configured to slide from the proximal position to the distal position to create a vacuum within the syringe barrel. The needle interface piece defines a needle channel communicating with the blood flash channel, the needle channel configured to receive a portion of the needle extending therethrough. The needle includes a notch extending through a wall of the needle to provide fluid communication between the needle lumen and the needle channel. The needle channel includes a first O-ring disposed annularly about the needle and disposed distally of the needle notch, and a second O-ring disposed annularly about the needle and disposed proximally of the needle notch, the first O-ring and the second O-ring each extending between an outer surface of the needle and an inner surface of the needle channel to provide a fluid-tight seal therebetween. The needle is slidably engaged with the needle channel. In some embodiments, a portion of the blood flash channel is formed of a flexible tube. The catheter is a central venous catheter or a rapid insertion central catheter.

### BRIEF DESCRIPTION OF DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1A shows a perspective view of an exemplary catheter insertion system, in accordance with embodiments disclosed herein.

FIG. 1B shows a side view of an exemplary catheter insertion system, in accordance with embodiments disclosed herein.

FIG. 1C shows a plan view of an exemplary catheter insertion system, in accordance with embodiments disclosed herein.

FIG. 2 shows an exploded view of an exemplary catheter insertion system, in accordance with embodiments disclosed herein.

FIG. 3A shows a side, cut away view of an exemplary catheter insertion system, in accordance with embodiments disclosed herein.

FIG. 3B shows a cross-section view of a needle interface of the catheter insertion system of FIG. 3A, in accordance with embodiments disclosed herein.

FIGS. 4A-4C show schematic views of a blood flash system for an exemplary catheter insertion system, in accordance with embodiments disclosed herein.

FIG. 5 shows a perspective view of an exemplary catheter insertion system, in accordance with embodiments disclosed herein.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal end portion” of, for example, a catheter disclosed herein includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal end portion, or the proximal length of the catheter can include the proximal end of the catheter; however, the proximal portion, the proximal end portion, or the proximal length of the catheter need not include the proximal end of the catheter. That is, unless context suggests otherwise, the proximal portion, the proximal end portion, or the proximal length of the catheter is not a terminal portion or terminal length of the catheter.

With respect to “distal,” a “distal portion” or a “distal end portion” of, for example, a catheter disclosed herein includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal end portion, or the distal length of the catheter can include the distal end of the catheter; however, the distal portion, the distal end portion, or the distal length of the catheter need not include the distal end of the catheter. That is, unless context suggests otherwise, the distal portion, the distal end portion, or the distal length of the catheter is not a terminal portion or terminal length of the catheter.

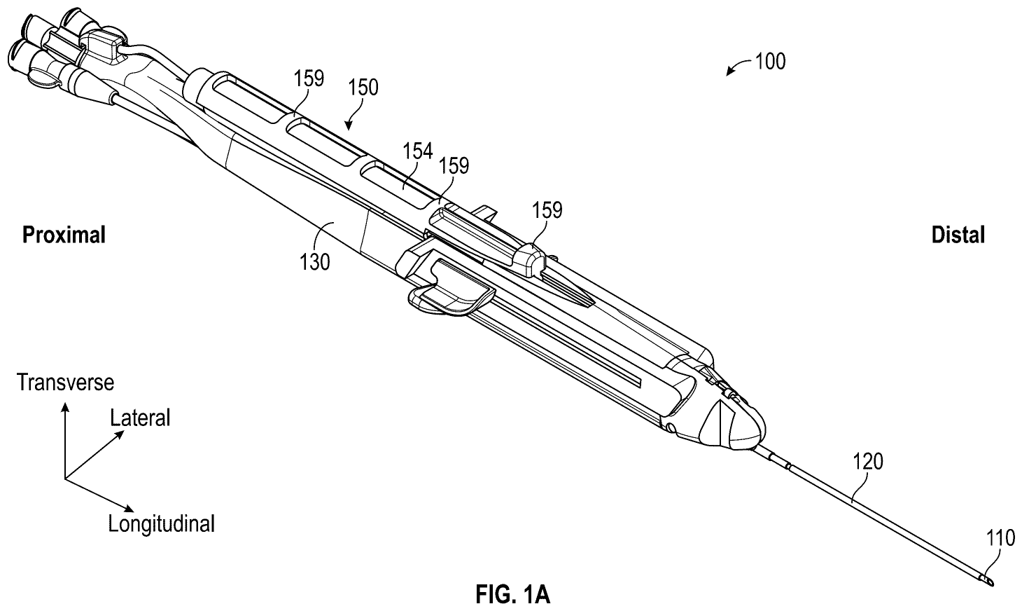

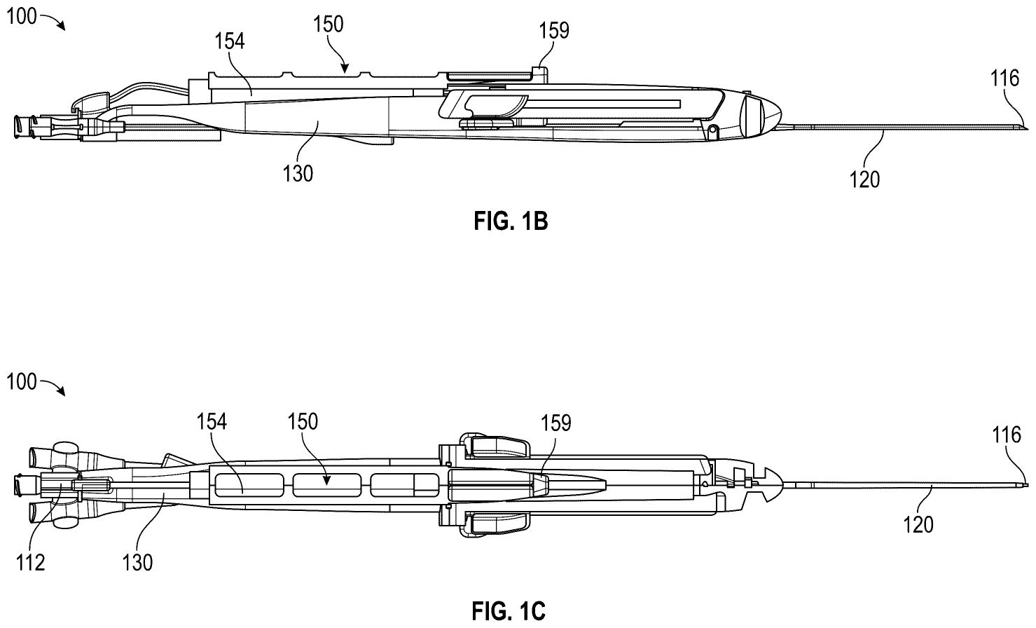

To assist in the description of embodiments described herein, as shown in FIG. 1A, a longitudinal axis extends substantially parallel to an axial length of the catheter. A lateral axis extends normal to the longitudinal axis, and a transverse axis extends normal to both the longitudinal and lateral axes. Unless defined otherwise, all technical and scientific terms used herein have the same meaning as commonly understood by those of ordinary skill in the art.

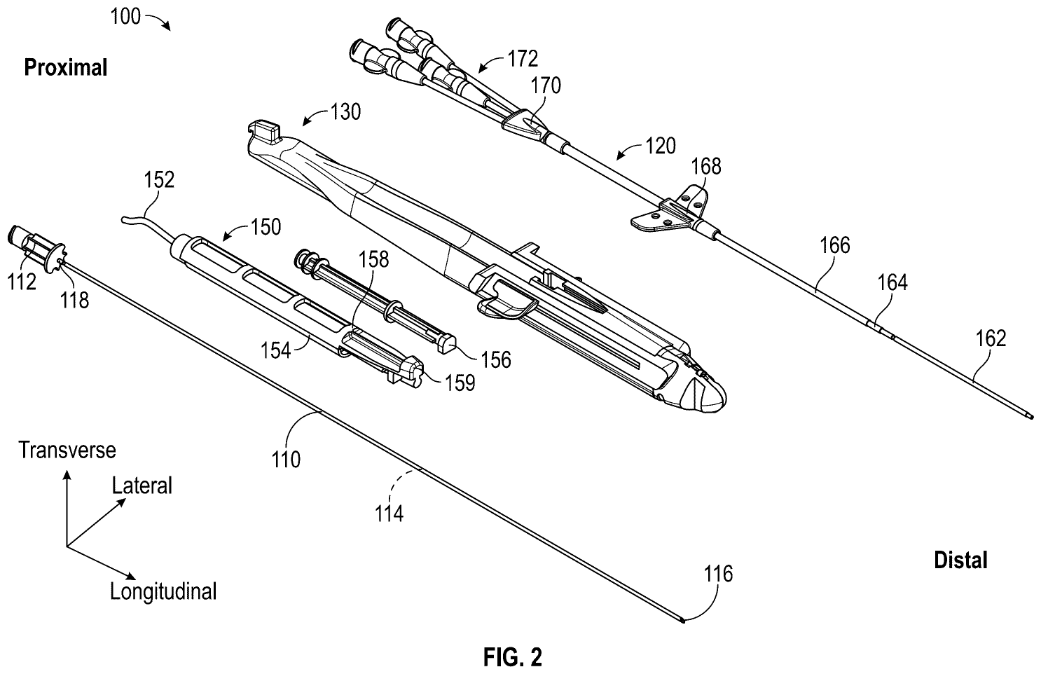

FIGS. 1A-2 show an exemplary catheter insertion system (“system”) 100, generally including a needle 110, a catheter 120, a housing 130, and a blood flash indicator 150. Optionally, the insertion system 100 can include one or more guidewires (not shown). The needle 110 can define a needle lumen 114 and can be supported by a needle hub 112 disposed at a proximal end thereof. The needle 110 can be configured to extend through at least a portion of a lumen of the catheter 120. A distal tip 116 of the needle 110 can extend distally of the distal tip of the catheter 120 and can define a sharpened tip configured for skin puncture and vascular access.

The catheter 120 can be a central venous catheter (CVC), a rapid insertion central catheter (RICC), or similar elongate catheter configured to provide access to a vasculature of a patient. As shown in FIG. 2, the catheter 120 can be a RICC catheter 120 including an access section 162 defining a first diameter, a dilation section 164, and a catheter body section 166 defining a second diameter, larger than the first diameter.

The access section 162 can define a single lumen and can be formed of a harder durometer material relative to the catheter body section 166. The catheter body section 166 can define one or more lumen and can be formed of a softer, more compliant material relative to the access section 162. The dilation section 164 can be formed of either the same material as the access section 162, or of a third material. The third material can be of a harder durometer relative to the material of the catheter body section 166. The dilation section 164 can provide a tapered transition between the first diameter of the access section 162 and the second diameter of the catheter body section 166. The access section 162 and the dilation section 164 can provide relatively more rigid mechanical properties and can be relatively more resistant to kinking or collapsing when an axial force is applied thereto, relative to the catheter body section 166. The catheter body section 166 can be relatively more compliant to facilitate negotiating tortuous vascular pathways. In an embodiment, the catheter 120 can further include a hub 168, a bifurcation 170, and/or one or more extension legs 172 each communicating with a lumen of the catheter 120. In an embodiment, the needle 110 can extend through an extension leg 172, through a lumen of the catheter body section 166 and through a lumen of the access section 162 to extend distally of a distal tip of the catheter 120.

In use, a clinician can access a vasculature using the RICC catheter 120 by insertion a needle tip 116 and a distal portion of the access section 162 into a vasculature. A blood flow can flow proximally through the needle lumen 114 to a blood flash indicator 150. A color and pulsatile flow can be observed to confirm correct vascular access. In case of incorrect vascular access, the access section 162 can be withdrawn and the insertion site closed by applying pressure, due to the relatively small diameter of the access section 162. Where correct vascular access is confirmed, the catheter 120 can be advanced, optionally over a guidewire, until a dilation section 164 enters the insertion site and dilates the insertion site to the second diameter of the catheter body section 166. The catheter body section 166 can then be advanced until a distal portion of the catheter 120 is at a target location within the vasculature. Further details of RICC catheters, associated insertion systems and associate methods thereof can be found in U.S. Pat. No. 10,376,675; U.S. Patent Publications U.S. 2019/0255294, U.S. 2021/0069471, U.S. 2021/0085927, U.S. 2021/0113809, U.S. 2021/0113810, U.S. 2021/0121661, U.S. 2021/0121667, U.S. 2021/0228843, U.S. 2021/0322729, U.S. 2021/0330941, U.S. 2021/0330942, and U.S. 2021/0361915, each of which are incorporated by reference in its entirety into this application.

As will be appreciated, depending on the configuration of the catheter insertion system, a blood flash flow would have to travel through the needle lumen 114, from a needle tip 116, substantially to a proximal end of the catheter 120 before exiting the catheter 120 and flowing distally to a blood flash indicator. Embodiments disclosed herein are directed to reducing the distance of travel between the needle tip 116 and the blood flash indicator 150.

In embodiments where the blood flash indicator is disposed towards the distal end of the device, the fluid path can be reduced by positioning the blood flash indicator 150 proximally. More specifically by placing the blood flash indicator 150 proximally of the catheter 120 center-of-mass, the length of the blood flash fluid path can reduced by between 20%-30%.

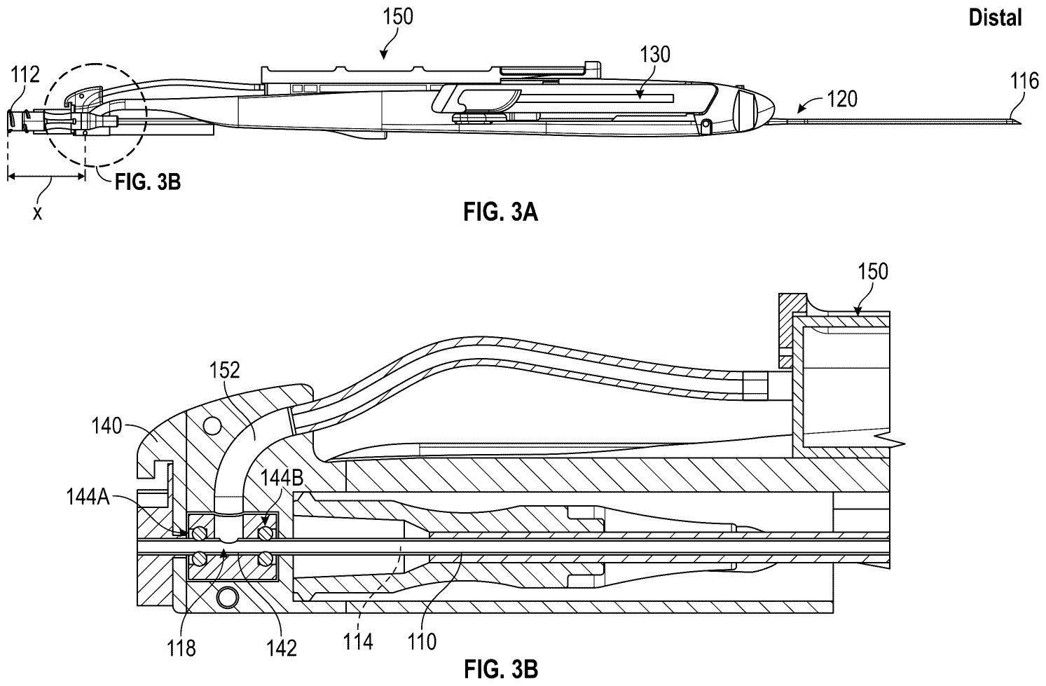

In an embodiment, as shown in FIGS. 3A-3B, the catheter insertion system 100 can include a needle interface 140 configured to provide fluid communication between the needle lumen 114 and the blood flash indicator 150, by way of a notch 118 disposed in a wall of the needle 110. The needle notch 118 can be positioned distally of the proximal end of the needle hub 112 by a distance (x). As such, the fluid path distance between the needle tip 116 and the blood flash indicator 150 can be further reduced by a distance (2x).

The needle interface 140 can define a needle channel 142 extending along a longitudinal axis and configured to receive a portion of the needle 110 therethrough. The needle 110 can be slidably engaged with the needle channel 142. The needle interface 140 can further include one or more O-rings 144 extending annularly about the needle 110 and disposed between an outer surface of the needle 110 and an inner surface of the channel 142, to provide a fluid tight seal therebetween.

The needle interface 140 can further include a blood flash channel 152 communicating between the needle channel 142 and the blood flash indicator 150. The blood flash channel 152 can extend substantially perpendicular from the needle channel 142 before extending towards the blood flash indicator 150. Prior to use, the needle notch 118 can be disposed within the needle channel 142, between a first O-ring 144A and a second O-ring 144B, and align with the blood flash channel 152. As the needle tip 116 is advanced into a vasculature, a blood flow can flow proximally through the needle lumen 114 through the notch 118 and into the blood flash channel 152 to the blood flash indicator 150. The first O-ring 144A and the second O-ring 144B can prevent a fluid flow from leaking between an outer surface of the needle 110 and an inner surface of the needle channel 142. The blood flow color or pulsatile flow can be observed to confirm correct vascular access. The needle 110 can then be withdrawn proximally through the needle channel 142 and, optionally, removed. In an embodiment, a portion of the needle 110 that is distal of the needle notch 118 can occlude the opening of the blood flash channel 152 with the needle channel 142 to seal the fluid therein.

In an embodiment, the blood flash indicator 150 can include a container configured to receive a blood flow therein. The container can be formed of a transparent material to allow a user to observe a color and pulsatile flow disposed therein. In an embodiment, the blood flash indicator 150 can include a vacutainer configured to maintain a vacuum therein to facilitate drawing a blood flow proximally through the needle lumen 114, through the needle notch 118, and into the vacutainer. In an embodiment, the vacuum can be maintained within the vacutainer by a portion of the needle 110 occluding the blood flash channel 152. When a user is ready to check for vascular access, the needle 110 can be advanced, relative to the housing 130, until the needle hub 112 abuts against the housing 130 and aligns the needle notch 118 with the blood flash channel 152. As such, the vacuum of the vacutainer is then placed in fluid communication with the needle lumen 114 and can facilitate drawing a blood flow proximally through the needle lumen 114.

In an embodiment, the blood flash indicator 150 can include a syringe barrel 154 and a plunger 156, slidably engaged therewith and configured to create a vacuum to draw a blood flow proximally through the needle lumen 114 and into the syringe barrel 154. In an embodiment, the operation of the syringe barrel 154 and plunger 156 can be reversed. For example, the plunger 156 can be fixedly engaged with the housing 130 to prevent any longitudinal movement relative thereto. The plunger 156 can be engaged with the housing 130 with an interference fit, snap-fit, press-fit engagement, adhesive, weld, bonding, or the like. The syringe barrel 154 can be supported by a barrel cradle 158. The barrel 154 and barrel cradle 158 assembly can be slidable relative to the plunger 156 and housing 130 assembly, and configured such that sliding the barrel 154 and cradle 158 assembly proximally can create a vacuum within the barrel 154. In an embodiment, the barrel cradle 158 can be formed of a transparent material to facilitate observing a blood flow therein.

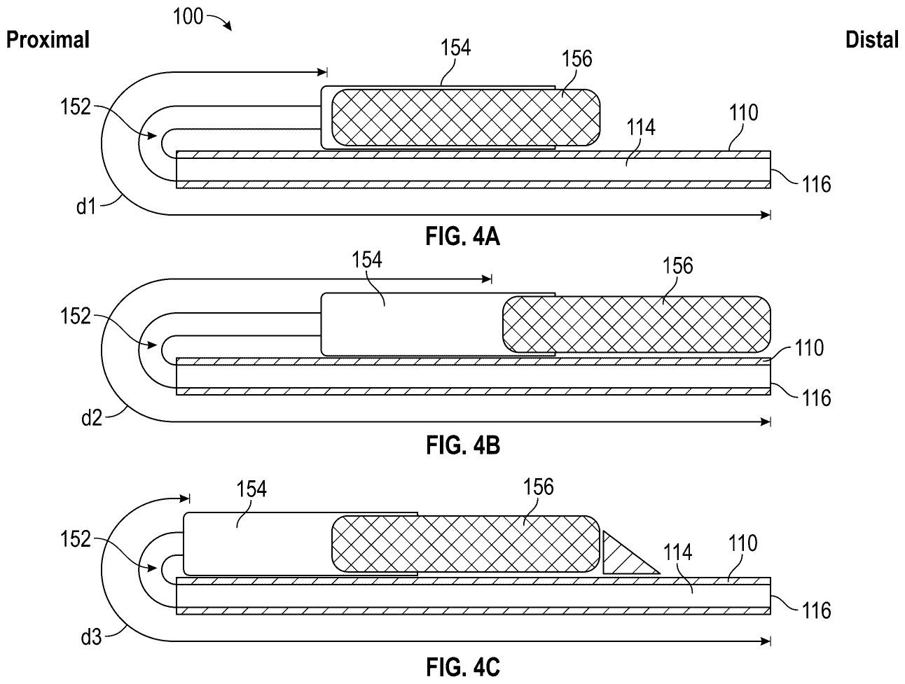

Advantageously, reversing the operation of the blood flash indicator syringe barrel 154 and plunger 156 can further reduce the fluid path between the needle tip and the blood flash indicator 150. For example, as shown in FIGS. 4A-4C, from a starting position shown in FIG. 4A, a blood flash fluid path can define a first distance (d1) extending from a needle tip 116 to a syringe barrel 154. As shown in FIG. 4B, where a blood flash indicator 150 includes a syringe barrel 154 and plunger 156 configured in a “conventional” way, the plunger 156 is withdrawn distally from the syringe barrel 154 to create the vacuum. However, in doing so the fluid path is extended from the first distance (d1) to a second distance (d2), which is greater than the first distance (d1).

By contrast, as shown in FIG. 4C, embodiments disclosed herein fix the plunger 156 to prevent any movement along a longitudinal axis, and the syringe barrel 154 slides proximally to create the vacuum. As such, a length of the fluid path is reduced from the first distance (d1) to a third distance (d3) that is less than the first distance (d1). By reversing the operation of the syringe blood flash indicator 150, the syringe barrel 154 can be slid proximally, towards the needle notch 118, to create the vacuum and thereby reducing a length of fluid path between the blood flash indicator 150 and the needle tip 116.

Advantageously, the action of the syringe barrel 154 and plunger 156 still allows a clinician to leverage the tactile and visual feedback offered via syringe-based blood flashback systems. Further, moving the syringe barrel 154 proximally moves the barrel 154 away from the distal end of the insertion device 100 providing a clearer line of sight at the insertion site and allows for additional operations to occur, for example the manipulation of guidewire advancement assemblies, catheter advancement assemblies, hinging housing portions, or the like.

In an embodiment, the barrel cradle 158 can include one or more gripping features 159 to facilitate grasping the syringe barrel 154 and urging the barrel 154 proximally. The gripping features 159 can include one or more abutments, finger loops, finger pad, ridges, ribs, or include materials having an increased friction co-efficient such rubber or silicone. In an embodiment, a portion of the blood flash channel 152, which provides fluid communication between the needle notch 118 and the syringe barrel 154, can include a flexible tube, or the like. Advantageously, the flexible tube can allow the syringe barrel 154 to slide proximally relative to the needle interface 140.

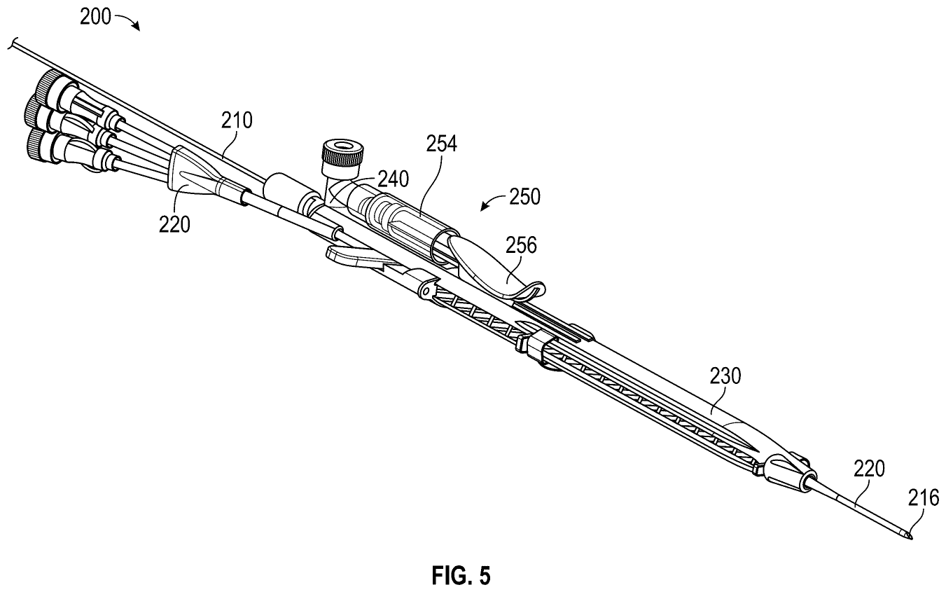

In an embodiment, as shown in FIG. 5, an insertion system 200 can include a blood flash indicator system 250 that includes a syringe used in a “conventional” manner. The insertion system 200 can generally include a housing 230, a catheter 220 (e.g. a CVC catheter, or a RICC catheter), and a needle 210. Optionally the insertion system 200 can include one or more guidewires (not shown). The blood flash indicator system 250 can include a syringe barrel 254 coupled with a housing 230 to prevent relative longitudinal movement therebetween. A plunger 256 can be slidably engaged with the syringe barrel 254 and can be advanced distally to create a vacuum within the syringe barrel 254. As shown, the plunger 256 can include a finger pad to facilitate gripping of the plunger. A proximal end of the syringe barrel can be aligned adjacent with a needle notch (not shown) and needle interface 240, as described herein.

In use, the insertion system 200 can be advanced, until a needle tip 216 enters a vasculature of a patient. The plunger 256 can extend distally to allow a user to manipulate the plunger 256 while grasping a distal portion of the housing 230. Advancing the plunger 256 distally can create a vacuum within the syringe barrel 254 and draw a blood flow through the needle lumen to the needle notch, through a needle interface 240, through the blood flash channel and into the syringe barrel 254. As shown the syringe barrel 254 can be formed of a transparent material to facilitate observing a blood flow color and pulsatile flow. Advantageously, longitudinally aligning the proximal end of the syringe barrel 254 adjacent to the needle interface 240, the length of the blood flash channel can be reduced, further reducing the overall length of the blood flash fluid path.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. A method of inserting a catheter into a vasculature of a patient, comprising:
disposing the catheter into a housing, wherein a distal portion of the catheter extends from a distal end of the housing, the housing including a blood flash indicator, the blood flash indicator comprising:
a syringe plunger fixed to the housing; and
a syringe barrel slidable relative to the syringe plunger;
positioning the housing adjacent the vasculature;
inserting a needle into the housing and through a lumen of the catheter until a distal tip of the needle extends from a distal end of the catheter;
accessing the vasculature via the distal tip of the needle; and
sliding the syringe barrel in a proximal direction relative to the syringe plunger to create a vacuum in a lumen of the needle to thereby draw blood through the lumen of the needle and into the syringe barrel.

2. The method according to claim 1, wherein inserting the needle into the housing comprises aligning a notch in a proximal end of the needle with a blood flash channel in fluid communication with the syringe barrel.

3. The method according to claim 2, wherein aligning the aperture in the proximal end of the needle comprises abutting a hub of the needle against the housing.

4. The method according to claim 2, wherein the blood flash indicator further comprises a vacutainer in fluid communication with the blood flash channel.

5. The method according to claim 2, wherein inserting the needle into the housing comprises inserting the needle into a needle channel of the housing, the needle channel of the housing including a first O-ring and a second O-ring spaced apart from the first O-ring, wherein aligning the notch comprises positioning the notch between the first O-ring and the second O-ring.

6. The method according to claim 2, wherein a portion of the blood flash channel is formed of a flexible tube.

7. The method according to claim 1, wherein the housing includes a cradle accommodating the blood flash indicator, the cradle including a gripping feature, further comprising grasping the gripping feature to maintain stability of the housing when sliding the syringe barrel in a proximal direction.

8. The method according to claim 1, further comprising determining proper access of the vasculature by observing a blood flow color or pulsatile flow in the syringe barrel.
